(19) United States
(12) Patent Application Publication
(10) Pub. No.: US 2013/0330229 A1
(43) Pub. Date: Dec. 12, 2013

(54) PLASMA SYSTEM FOR AIR STERILIZATION

(71) Applicants: Gregory Fridman, Philadelphia, PA (US); Alexander Fridman, Philadelphia, PA (US); Yurii V. Mukhin, Altadena, CA (US); Alexander F. Gutsol, San Ramon, CA (US); Nachiket Vaze, Mumbai (IN)

(72) Inventors: Gregory Fridman, Philadelphia, PA (US); Alexander Fridman, Philadelphia, PA (US); Yurii V. Mukhin, Altadena, CA (US); Alexander F. Gutsol, San Ramon, CA (US); Nachiket Vaze, Mumbai (IN)

(73) Assignee: Drexel University, Philadelphia, PA (US)

(21) Appl. No.: 13/637,466

(22) PCT Filed: Mar. 30, 2011
(86) PCT No.: PCT/US11/30483

§ 371 Date: May 9, 2013

Related U.S. Application Data
(60) Provisional application No. 61/319,356, filed on Mar. 31, 2010.

(51) Int. Cl. A61L 2/14

(52) U.S. Cl. CPC A61L 2/14
USPC 422/4; 422/186; 422/186.3

(57) ABSTRACT

A method for decontaminating bioaerosol with high concentrations of bacterial, viral, spore and other airborne microorganisms or biologic contaminants in flight at high flow rates. A plasma screen created across the flow of air contaminated with airborne biologic agents renders contaminants non-culturable within milliseconds. The technology may cooperate with heating, ventilation, and air conditioning (HVAC) systems. It may be particularly beneficial in preventing bioterrorism and the spread of toxic or infectious agents, containing airborne pandemic threats such as avian flu, sterilizing spaces such as hospitals, pharmaceutical plants and manufacturing facilities, treating exhaust ventilation streams, minimizing biological environmental pollutants in industrial settings, improving general air quality, preventing sick building syndrome.

### CROSS-REFERENCE TO RELATED APPLICATIONS

This application claims the benefit of priority to U.S. Provisional Patent Application Ser. No. 61/319,356, filed Mar. 31, 2010, which is herein incorporated by reference in its entirety for all purposes.

### FIELD OF THE INVENTION

The disclosed inventions are in the field of decontaminating high concentrations of bacterial bioaerosols, viral bioaerosols, and other airborne microorganisms in flight at high flow rates using plasma. The disclosed inventions are particularly applicable to the Heating, Ventilation and Air Conditioning (HVAC) industry, hospitals, food processing plants, and bioterrorism defense industry.

### BACKGROUND OF THE INVENTION

The escalating threat of airborne biologic and bioterrorism agents present a need for robust technologies and methods to mitigate the spread of airborne contaminants. The avian flu pandemic, the 1976 Legionnaires outbreak in Philadelphia, and the 2001 anthrax terrorism in the United States demonstrate the ability to rapidly spread biologic contaminants through ventilation systems. To address these concerns, there is a continuing need to deactivate microorganisms, such as viruses and bacteria, in solution and on surfaces.

Plasmas, referred to as the “fourth state of matter”, are ionized gases having at least one electron that is not bound to an atom or molecule. In recent years, plasmas have become of significant interest to researchers in fields such as organic and polymer chemistry, fuel conversion, hydrogen production, environmental chemistry, biology, and medicine, among others. This is, in part, because plasmas offer several advantages over traditional chemical processes. For example, plasmas can generate much higher temperatures and energy densities than conventional chemical technologies; plasmas are able to produce very high concentrations of energetic and chemically active species, and plasma systems can operate far from thermodynamic equilibrium, providing extremely high concentrations of chemically active species while having a bulk temperature as low as room temperature.

Plasmas are generated by ionizing gases using any of a variety of ionization sources. Depending upon the ionization source and extent of ionization, plasmas may be characterized as either thermal or non-thermal. Thermal and non-thermal plasmas can also be characterized by the temperature of their components. Thermal plasmas are in a state of thermal equilibrium, that is, the temperature of the free electrons, ions, and heavy neutral atoms are approximately the same. Non-thermal plasmas, or cold plasmas, are far from a state of thermal equilibrium. The temperature of the free electrons is much greater than the temperature of the ions and heavy neutral atoms within the plasma.

Decontamination of microorganisms in flight using non-thermal plasma technology, however, has not been effectively implemented. Plasma-based air decontamination has only been found effective when coupled with high efficiency particulate air (HEPA) filters, which trap and kill microorganisms. HEPA filters, however, are inefficient at trapping submicron-sized airborne microorganisms. Moreover, HEPA filters also cause significant pressure losses in HVAC systems, generating high energy and maintenance costs. The filters function as a surface on which contaminants are captured. Therefore, the prior art methodologies are, in essence, the same as standard plasma surface sterilization. Numerous technologies similarly sterilize air by directing plasma emissions at a filter surface, which entraps the biologic contaminants.

Apart from treatments in solution or on surface, there remains a need to develop a means for in-flight plasma-based decontamination so as to be able to deactivate microorganisms in the air while in motion. The invention is directed to this and other important needs. This will be particularly useful for decontaminating or sterilizing air in ventilation systems and preventing the spread of airborne biologic agents.

### SUMMARY OF THE INVENTION

Provided herein are modular systems for inactivating biological agents in gaseous medium, comprising a series of fluidically-coupled non-thermal plasma generators, each of said non-thermal plasma generators capable of receiving a gaseous medium, contacting the gaseous medium with a non-thermal plasma to give rise to a plasma-treated gaseous medium, and discharging the plasma-treated gaseous medium. The plasma-treated gaseous medium of at least one of the non-thermal plasma generators is capable of being received by at least one other non-thermal plasma generator.

Also provided are modular systems for inactivating biological agents in a gaseous medium comprising a series of fluidically-coupled non-thermal plasma generators, each of said non-thermal plasma generators comprising an entrance port capable of receiving a gaseous medium, and an exit port capable of discharging plasma-treated gaseous medium from the plasma generator. The series of fluidically-coupled non-thermal plasma generators is configured such that at least one of the discharge ports of one non-thermal plasma generators is fluidically coupled to the entrance port of at least one other non-thermal plasma generator.

Also provided are methods for inactivating biologic agents in a gaseous medium, comprising directing the flow of the gaseous medium through a series of fluidically-coupled non-thermal plasma generators. The series of fluidically-coupled non-thermal plasma generators comprises a first non-thermal plasma generator and at least a second non-thermal plasma generator, to give rise to a plasma-treated gaseous medium. The plasma-treated gaseous medium of the first non-thermal plasma generator is discharged to the entrance of the second non-thermal plasma generators, and the plasma-treated gaseous medium is discharged from the last in the series of the fluidically-coupled non-thermal plasma generators.

Also disclosed are methods for inactivating biologic agents in a gaseous medium, comprising of parts directing a gaseous medium comprising biological agents through an entrance port of a dielectric barrier discharge device, contacting the gaseous medium with a non-thermal plasma generated by said dielectric barrier discharge device to give rise to a plasma-treated gaseous medium, directing the plasma-treated gaseous medium through an exit port of the dielectric barrier discharge device, and further directing the plasma-treated gaseous medium through an exit port of the dielectric barrier discharge device, and further directing the plasma-treated gaseous medium through an entrance port of at least a second dielectric barrier discharge device.

The general description and the following detailed descriptions are exemplary and explanatory only and are not restrictive of the invention, as defined in the appended claims. Other aspects of the present invention will be apparent to those skilled in the art in view of the detailed description of the invention as provided herein.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention. However, the invention is not limited to the specific methods, compositions, and devices disclosed. In addition, the drawings are not necessarily drawn to scale.

FIG. 1 is a schematic of an embodiment of a suitable Pathogen Detection and Remediation Facility (PDRF).

FIG. 2 shows an embodiment of an individual plasma module. Each individual electrode can be removed to produce different configurations of plasma discharge.

FIG. 3 shows an embodiment of a complete plasma unit with individually replaceable units.

FIG. 4 shows an embodiment of a plasma sterilizer unit in vertical configuration, which includes a fan unit, a plasma module, and a filter for removal of excess ozone.

In FIG. 5 the particle count indicates that the spores are detectable inside the chamber and are not lost inside the system. No viable bacillus globigii is detected inside the chamber after 28 minutes of plasma treatment.

FIG. 6 shows that the maximum inactivation is at 8 minutes after injection, where the percentages as compared to initial concentration are 50% for control and 17% for Test. The results indicate an 83% reduction in viability in 8 minutes.

### DETAILED DESCRIPTION OF ILLUSTRATIVE EMBODIMENTS

The present subject matter may be understood more readily by reference to the following detailed description taken in connection with the accompanying figures and examples, which form a part of this disclosure. It is to be understood that this invention is not limited to the specific devices, methods, applications, conditions, or parameters described and/or shown herein, and that the terminology used herein is for the purpose of describing particular embodiments by way of example only and is not intended to be limiting of the claimed invention.

Also, as used in the specification, including the appended claims, the singular forms “a”, “an”, and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about”, it will be understood that the particular value forms another embodiment. All ranges are inclusive and combinable.

### Terms

As used herein, the term “non-thermal plasma” refers to an electrically neutral mixture of atoms, molecules, electrons and ions that cannot be described by one temperature. The average energy of electrons in non-thermal plasma is usually on the level of more than 1 electron-volt (eV), which corresponds to a temperature of about 11,600 K. By comparison, the average translational temperature of heavy particles (ions, molecules, and atoms) is usually less than 3,000 K, and is often very close to ambient temperature, or approximately 20° C.

As used herein, the term “biological agent” refers to an active biological agent if it is capable of reproduction or proliferation (culturable microorganisms) in a special appropriate media or in human organisms. If the microorganism is not able to reproduce itself, it is highly probable that it can not harm another organism even if its structure is mechanically intact, and thus such microorganisms are considered to be inactivated. The methods described herein can be used for sterilizing biologic contaminants entrained, dispersed, or suspended in a gaseous media at high flow rates by plasma emissions and a system for carrying out the method.

As used herein, a “Pathogen Detection and Remediation Facility (PDRF)” is a facility incorporating a plasma emission device such as a Dielectric Barrier Discharge (DBD) device or Magnetically-Rotating Gliding Arc (MRGA) device.

As used herein, the term “glow discharge” refers to a plasma source that generates a non-equilibrium plasma between two electrodes under a direct current. Fluorescent light is a common type of glow discharge. This glow discharge is established in a long tube with a potential difference applied between an anode at one end of the tube and a cathode at the other end. The tube is filled with an inert or reactive gas often under pressure. Due to the potential difference between the electrodes, electrons are emitted from the cathode and accelerate toward the anode. The electrons collide with gas atoms in the tube and form excited species. These excited species decay to lower energy levels through the emission of light. The ionized species generated by the collision of electrons with gas atoms travel toward the cathode and release secondary electrons, which are then accelerated toward the anode. This generation of electrons, referred to as secondary emission, is in contrast to the intensive formation of electrons at the surface of the cathode in thermal plasma generation.

As used herein, the term “gliding arc discharge” refers to a variation of the thermal arc discharge that provides for some of the benefits of the thermal plasmas (e.g., high energies, high plasma densities, etc.). It is formed by the application of a potential across two diverging electrodes. At the shortest distance between the electrodes (usually about 1 to 2 mm), an arc forms when the electric field between the electrodes is greater than about 3 kV/mm in air. Gas flow (typically about 10 m/s) is introduced into the reactor which pushes the arc downstream through the diverging electrodes. As the distance between the electrodes increases, the amount of energy loss to the surroundings can not be balanced by the supplied power. At this point, the plasma transforms from thermal to non-thermal and the temperature of the gas dramatically reduces. Gliding arc discharges have been used for fuel conversion, carbon dioxide conversion to carbon monoxide and oxygen, and surface treatments.

Plasmas are generated by ionizing gases using any of a variety of ionization sources. Plasmas may be characterized as either thermal or non-thermal, depending upon the ionization source and the extent of ionization. Thermal and non-thermal plasmas can also be characterized by the temperature of their components. Thermal plasmas are in a state of thermal equilibrium, meaning the temperature of the free electrons, ions, and heavy neutral atoms are approximately the same. Non-thermal plasmas are far from a state of thermal equilibrium; hence, the temperature of the free electrons is much greater than the temperature of the ions and heavy neutral atoms within the plasma.

The initial generation of free electrons may vary depending upon the ionization source. With respect to both thermal and non-thermal ionization sources, electrons may be generated at the surface of the cathode due to a potential applied across the electrode. In addition, thermal plasma ionization sources may also generate electrons at the surface of a cathode as a result of the high temperature of the cathode (thermionic emissions) or high electric fields near the surface of the cathode (field emissions).

The energy from these free electrons may be transferred to additional plasma components, providing energy for additional ionization, excitation, dissociation, etc. With respect to non-thermal plasmas, the ionization process typically occurs by direct ionization through electron impact. Direct ionization occurs when an electron of high energy interacts with a valence electron of a neutral atom or molecule. If the energy of the electron is greater than the ionization potential of the valence electron, the valence electron escapes the electron cloud of the atom or molecule and becomes a free electron according to: e⁻+A→A⁺+e⁻+e⁻.

As the charge of the ion increases, the energy required to remove an additional electron also increases. Thus, the energy required to remove an additional electron from A⁺ is greater than the energy required to remove the first electron from A to form A⁺. A benefit of non-thermal plasmas is that because complete ionization does not occur, the power to the ionization source can be adjusted to increase or decrease ionization. This ability to adjust the ionization of the gas provides for a user to “tune” the plasma to their specific needs.

An exemplary thermal plasma ionization source is an arc discharge. Arc discharges have been otherwise used for applications such as metallurgy, metal welding and metal cutting and are known per se. Arc discharges are formed by the application of a potential to a cathode and are characterized by high current densities and low voltage drops. Factors relevant to these characteristics are the usually short distance between the electrodes (typically a few millimeters) and the mostly inert materials of the electrodes (typically, carbon, tungsten, zirconium, silver, etc.). The majority of electrons generated in arc discharges are formed by intensive thermoionic and field emissions at the surface of the cathode. A much larger number of the electrons are generated directly from the cathode as opposed to secondary sources such as excited atoms or ions.

Because of this intense generation of electrons at the cathode, current at the cathode is high, which leads to Joule heating and increased temperatures of the cathodes. These high temperatures can result in evaporation and erosion of the cathode. The anode in arc discharges may be either an electrode having a composition identical or similar to the cathode or it may be another conductive material. For example, the anode in arc discharges used in metal welding or cutting is the actual metal to be welded or cut.

Although thermal plasmas are capable of delivering extremely high powers, they have several drawbacks. In addition to the electrode erosion problems discussed above, thermal plasmas have additional drawbacks. For example, thermal plasmas do not allow for adjusting the amount of ionization, they operate at extremely high temperatures, and they lack efficiency.

Non-thermal plasma ionization sources have alleviated some of the above-mentioned problems. Exemplary ionization sources for non-thermal plasmas include glow discharges, floating electrode dielectric barrier discharges, and gliding arc discharges, among others. In contrast to thermal plasmas, non-thermal plasmas provide for high selectivity, high energy efficiencies, and low operating temperatures. In many non-thermal plasma systems, electron temperatures are about 10,000 K while the bulk gas temperature may be as cool as room temperatures.

DBD may be created using an alternating current source at a frequency of from about 0.1 kHz to about 500 kHz between a high voltage electrode and a ground electrode. In addition, one or more dielectric barriers are placed between the electrodes. DBDs have been employed for over a century and have been used for the generation of ozone in the purification of water, polymer treatment, and for pollution control. DBDs prevent spark formation by limiting current between the electrodes.

Several materials can be utilized for the dielectric barrier. These include glass, quartz, aluminum nitride, and ceramics, among others. The clearance between the discharge gaps is typically between about 0.1 mm and several centimeters. The required voltage applied to the high voltage electrode varies depending upon the pressure and the clearance between the discharge gaps. For a DBD at atmospheric pressure and a few millimeters between the gaps, the voltage required to generate a plasma is typically about 10 kV. In certain embodiments, the ground electrode of the DBD may be an external conductive object, such as a human body. This is known as floating-electrode DBD (FE-DBD). FE-DBD has recently been utilized in medical applications.

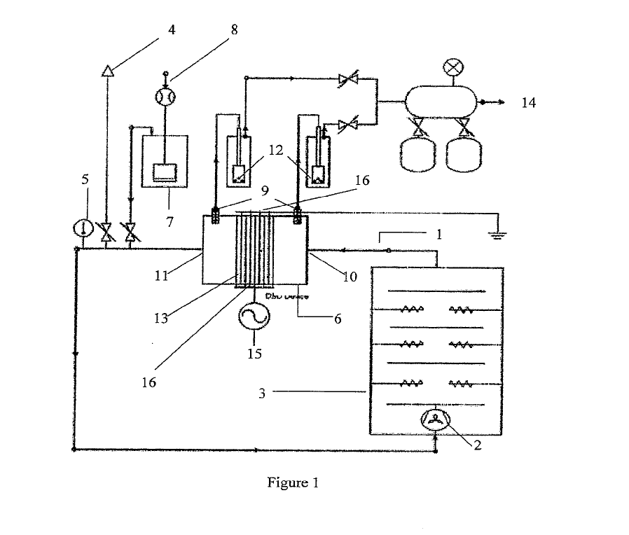

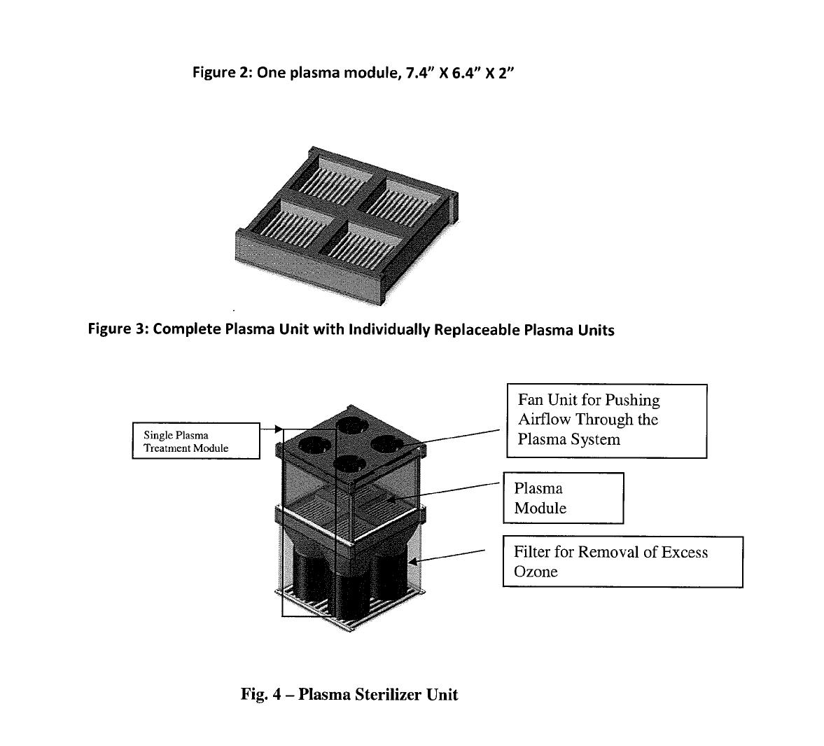

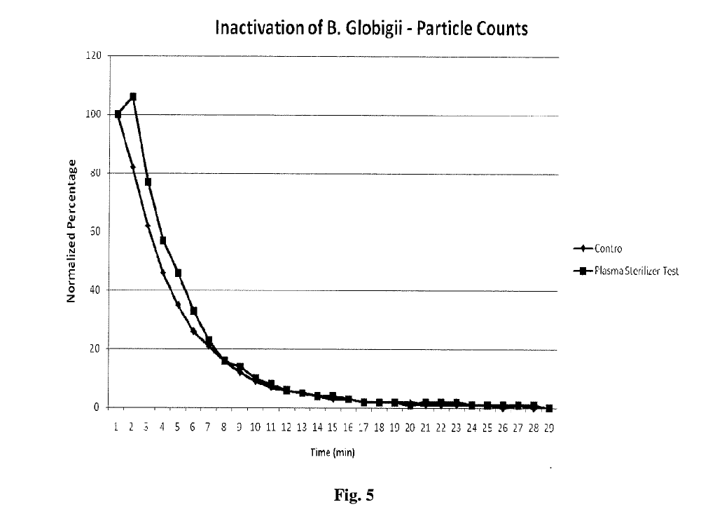

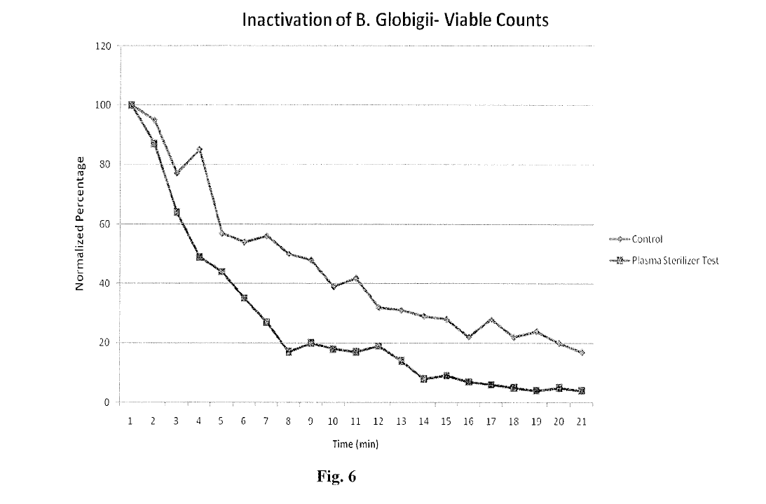

One suitable embodiment includes a PDRF and a plasma emission device, which renders biologic contaminants non-culturable. A suitable PDRF, as shown in FIG. 1, can be a plug flow reactor that is capable of circulating and sampling a gaseous media as well as bioaerosol generation, capture and containment. This PDRF is designed to operate at high airflow rates of about 25 L/s or greater, which are typical for indoor ventilation systems. A centrifugal blower 2 drives and circulates the contaminated gaseous media through a mixing chamber 3 and ventilation line 1. In a preferred embodiment, the PDRF is a closed system that allows for humidity control, which may be set to optimize sterilization capabilities. The air pressure and temperature within the line is regulated by a pressure release valve 4 and hygrometer/thermometer 5.

A suitable PDRF recirculating airflow system can repeatedly treat bioaerosols entrained, dispersed, or suspended in a gaseous media, recirculating through the plasma emission device 6, as shown in FIG. 1. The system also includes a bioaerosol nebulizer 7 and compressed air source 8 for the purposes of introducing a sample of biologic contaminants entrained, dispersed, or suspended in a gaseous media.

A suitable plasma emission device 6 may include any mechanism capable of producing plasma in a directed air or aerosol stream. In a preferred embodiment, the device may be a Dielectric Barrier Discharge ventilation grating (DBDG) plasma device or a Magnetically-Rotated Gliding Arc (MRGA) device. 1-mm wires 13 of one grating are covered with quartz capillaries of 2-mm outer diameter and connected to a high voltage AC power supply 15. There are 1.5-mm air gaps between these insulated wires and 1-mm bare wires 13 of the second grating that are grounded. When high-voltage AC power supply is on, non-equilibrium plasma is generated in the air gaps between bare and insulated wires, connected to a high voltage source 15, air sampling ports 9, connected to a set of liquid impingers 12 and a vacuum source 14.

A suitable DBD device 6 is used to generate plasma. The DBD is an alternating current discharge between two electrodes 16, at least one of which is covered by a dielectric. Various materials can be utilized for the dielectric barrier, including plastic, glass, quartz, and ceramics, among others.

DBD plasma can be formed in the gas filled area, otherwise known as the discharge gap, between one electrode and a dielectric or between two dielectrics. The clearance between discharge gaps is typically between about 0.01 mm to several centimeters.

The DBD is driven by an applied alternating high voltage, which generates a high electric field between the electrodes 16. The required voltage applied to the electrodes varies, depending upon the pressure and clearance between the discharge gaps. For DBD at atmospheric pressure and with only a few millimeters between the gaps, the voltage required to generate a plasma may vary, but in some configurations, is about 10 kV.

In the absence of a dielectric, the discharge starting from the first spark, would rapidly progress to an arc, as the electrons in the spark would initiate a series of ionization events, leading to very high current and ultimately to arc formation. The dielectric prevents arc formation by accumulating charge on the surface and generating an electric field that opposes the applied field, thereby limiting the current and preventing development of an uncontrolled discharge. Alternating high voltage polarities ensures formation of this discharge in each half of the voltage cycle.

Typically, DBD devices 6 operate in the kilohertz range, so plasma between the electrodes 16 does not have enough time to extinguish completely. In one embodiment, the non-thermal plasma discharge is generated by a high frequency pulsed or continuous voltage of from about 1 to about 20,000 kHz, optionally, from about 5 to about 30 kHz, and a peak-to-peak voltage of about 1 to about 50 kV, optionally, from about 5 to about 30 kV.

In certain embodiments, the DBD may be generated using an alternating current at a frequency of from about 0.1 kHz to about 500 kHz between a high voltage electrode and a ground electrode. It should be noted that in certain configurations, a single pulse may be used. Therefore, the present subject matter may be preferably used in applications ranging from a single pulse to a series of pulses operating at frequencies up to about 500 kHz.

As gaseous media in which biologic contaminants are entrained, dispersed or suspended is introduced into the DBD device 6 through an entry port 10, a quasi-sinusoidal waveform is generated by a quasi-pulsed high-voltage source and applied across the electrode gaps generating a high electric field and non-equilibrium plasma that covers the whole area between electrodes 16. The period between pulses is approximately 600 μs, peak-to-peak voltage is 28 kV, and current reaches nearly 50 amps in a pulse.

The gaseous medium may be contacted by the non-thermal plasma for different periods of time. The period of time may vary depending upon factors such as the composition of the gaseous medium, the type of plasma, and the plasma intensity. In certain embodiments, the gaseous medium may be contacted with the non-thermal plasma for at least about 10 milliseconds, or at least about 60 milliseconds, or for at least about 90 milliseconds. The duration of the contact between the gaseous medium and the non-thermal plasma may be referred to as “hold time”. In certain embodiments, the hold time may be at least about 5 seconds, or at least about 30 seconds, or at least about 60 seconds, or at least about 600 seconds.

The average power of a suitable discharge can be in the range of from about 50 Watts to about 1,000 Watts, and considering the discharge area in the range of from about 91 cm² to about 500 cm², the power density can be in the range of from about 1 Watts/cm² to about 6 Watts/cm². The majority of power is discharged in the very short duration of the pulse itself, which has a period of 77 μs and average pulse power of 2,618 W. Since the residence time of a bioaerosol particle passing through the discharge area is 0.73 ms and the period between pulses is 0.6 ms, this means that each bioaerosol particle that passes through the DBD discharge experience about 1 pulse of DBD discharge power, assuming the discharge is fairly uniform and gaps between streamers are not considered. The air passes through the plasma stream and leaves the DBD through an exit port 11.

Suitably, a plasma “curtain” is created, and should not have large holes, e.g. larger than the distance between DBD surfaces. The time between high voltage pulses that generate plasma should not be significantly larger than the residence time of air in plasma, so that t=Sd/Q, where S is the free area where plasma is generated, d is the thickness of the bare electrodes and Q is the air flow rate.

The power should be sufficient to provide the desired degree of decontamination. The extent of decontamination depends upon factors such as the type and amount of organic material, plasma energy, and hold time. In certain embodiments, the gaseous medium is at least about 50% decontaminated upon contact with the decontamination composition, at least about 75% decontaminated, at least about 90% decontaminated, or at least about 95% decontaminated. However, the present invention shows that a modular system greatly increases the decontamination percentage of the gaseous medium.

The modular system further comprises one or more subsystems disposed in series with at least one non-thermal plasma generator. The subsystem may be comprised of a water mist injector, a heater, a filter, an organic vapor injector, a manganese dioxide/copper oxide based catalyst ozone filter, or a heat exchanger. In addition, at least one of the non-thermal plasma generators may be a DBD device.

Additional subsystems may be connected to the DBD device, including a water mist injector, a heater, a filter, a vapor injector, a manganese dioxide/copper oxide based catalyst ozone filter, or a heat exchanger. In addition, at least one of the non-thermal plasma generators may be a DBD device. The DBD device is capable of generating a high frequency plasma of about 1 kHz to about 20,000 kHz. In a preferred embodiment, the DBD device is capable of generating a high frequency plasma of about 5 kHz to about 30 kHz.

The non-thermal barrier DBD is generated by a pulsed or continuous frequency of about 1 kHz to about 20,000 kHz. In a preferred embodiment the frequency is from about 5 kHz to about 30 kHz. In a more preferred embodiment, the frequency is from about 1 kV to about 50 kV. In the most preferred embodiment, the frequency is from about 5 kV to about 30 kV.

### Examples

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### Example 1

A PDRF system for a bioaerosol treatment facility is designed to provide a recirculating gaseous media environment. The PDRF system has a total volume of 250 liters and is designed to operate at high airflow rates of at least 25 L/s, which is typical of indoor ventilation systems. The system has an inlet with an attached Collison nebulizer for bioaerosol generation and two air-sampling ports connected to a vacuum air sampling system. The system also has a large mixing chamber that contains a series of aluminum baffle plates and a variable speed centrifugal blower motor that drives the air through the DBD treatment chamber. The residence time, defined as the time for one bioaerosol particle to make one complete revolution through the system, is approximately 10 seconds.

The DBD device may include a thin plane of wires with equally spaced air gaps of 1.5 mm, and each second wire is a high voltage electrode. The high voltage electrodes are about 1 mm diameter copper wires shielded with a quartz capillary dielectric that has an approximate wall thickness of 0.5 mm. The total area of the DBD, including electrodes is 214.5 cm² and without electrodes is 91.5 cm². The DBD device further has two air sample ports located at a distance of 10 cm from each side of the discharge area so that bioaerosol can be sampled immediately before and after it enters the plasma discharge. When the PDRF system is operated at a flow rate of 25 L/s, the air velocity inside the DBD chamber is 2.74 m/s, and the residence time of one bioaerosol particle containing one E. Coli bacterium, passing through the DPD is approximately 0.73 milliseconds.

The DBD device was operated using a quasi-pulsed power supply 15 that delivers a quasi-sinusoidal voltage waveform with a very fast rise time that nearly simulates a true square wave pulse. The period between pulses was approximately 600 μs, peak-to-peak voltage is 28 kV, and current pulses that passed through air plasma reached 50 amps. The average power of the discharge is approximately 330 watts. The average discharge area is 91 cm², and the average power density was 3.6 watts/cm². The majority of power was discharged in the very short duration of the pulse itself, which had a period of 77 μs and average pulse power of 2618 watts. Since the residence time of a bioaerosol particle passing through the discharge area was 0.73 ms and the period between pulses was 0.6 ms, assuming the discharge was fairly uniform, and there were no gaps between streamers, each bioaerosol particle that passed through the DBD experienced about 1 pulse of DBD power.

### Example 2

The spores used for testing the system were Bacillus globigii (BG) spores, donated by the U.S. Department of Defense (Dugway Proving Ground, Utah). Stock concentration powder was approximately 1×10¹¹ cfu/gm.

The plasma sterilizer was placed inside the room between the injection point and the air sampler. The plasma sterilizer was turned ON remotely from inside the control room. The spores were then injected into the air. Samples were taken each minute for 30 minutes. The results were analyzed using plate count method.

### Example 3

The average power of a suitable discharge was about 330 W and considering the discharge area of 91 cm−2, the power density was 3.6 W/cm−2. The majority of power was discharged in the very short duration of the pulse itself, which had a period of 77 μs and average pulse power of 2618 W. Since the residence time of a bioaerosol particle passing through the discharge area was 0.73 ms, and the period between pulses was 0.6 ms, each bioaerosol particle that passed through the DBD discharge experienced about 1 pulse of DBD discharge power.

Having described the preferred embodiments of the invention which are intended to be illustrative and not limiting, it is noted that modifications and variations can be made by persons skilled in the art in light of the above teachings. It is therefore to be understood that changes may be made in the particular embodiments of the invention disclosed which are within the scope and spirit of the invention as outlined by the appended claims. Having thus described the invention with the details and particularity required by the patent laws, the intended scope of protection is set forth in the appended claims.

What is claimed:

1. A modular system for inactivating biological agents in a gaseous medium, comprising:
a series of fluidically-coupled non-thermal plasma generators, each of said non-thermal plasma generators capable of the following:
receiving a gaseous medium; contacting a gaseous medium with a non-thermal plasma to give rise to a plasma-treated gaseous medium; and discharging said plasma-treated gaseous medium,
wherein the plasma-treated gaseous medium of at least one of the non-thermal plasma generators is capable of being received by at least one other non-thermal plasma generator.

2. The modular system of claim 1 further comprising one or more sub-systems disposed in series with at least one of said non-thermal plasma generators.

3. The modular sub-system of claim 2, wherein said sub-system comprises a water mist injector sub-system.

4. The sub-system of claim 2, wherein said sub-system comprises a heater.

5. The sub-system of claim 2, wherein said sub-system comprises a filter.

6. The sub-system of claim 2, wherein said sub-system comprises an organic vapor injector.

7. The sub-system of claim 2, wherein said sub-system comprises a manganese dioxide/copper oxide ozone filter.

8. The sub-system of claim 2, wherein said sub-system comprises a heat exchanger.

9. The modular system of claim 1, wherein at least one of said non-thermal plasma generators is a dielectric barrier discharge device.

10. A modular system for inactivating biological agents in a gaseous medium, comprising:
a series of fluidically-coupled non-thermal plasma generators, each of said non-thermal plasma generators comprising the following:
an entrance port capable of receiving a gaseous medium; and
an exit port capable of discharging plasma-treated gaseous medium from the plasma generator,
wherein the series of fluidically-coupled non-thermal plasma generators is configured such that at least one of the discharge ports of one non-thermal plasma generators is fluidically coupled to the entrance port of at least one other non-thermal plasma generator.

11. The modular system of claim 10, wherein at least one of said non-thermal plasma generators is a dielectric barrier discharge device.

12. The modular system of claim 11, in which one or more sub-systems are connected to the dielectric barrier discharge device.

13. The sub-system of claim 12, wherein of the sub-system includes a water mist injector sub-system, configured to inject water mist into one or more of the gaseous mediums.

14. The sub-system of claim 12, wherein said sub-system includes a heater, configured for heating the gaseous medium.

15. The sub-system of claim 12, wherein said sub-system includes a filter.

16. The sub-system of claim 12, wherein said sub-system includes an organic vapor injector for injecting organic vapor into the gaseous medium.

17. The sub-system of claim 12, wherein said sub-system includes a UV or carbon ozone destroyer.

18. The sub-system of claim 12, wherein said sub-system includes a heat exchanger for heating and cooling the gaseous medium.

19. The modular system of claim 11, wherein said dielectric barrier discharge device is capable of generating a high frequency plasma of about 1 kHz to about 20,000 kHz.

20. The modular system of claim 11, wherein said dielectric barrier discharge device is capable of generating a high frequency plasma of about 5 kHz to about 30 kHz.

21. A method for inactivating biologic agents in a gaseous medium, comprising:
directing the flow of a gaseous medium through a series of fluidically-coupled non-thermal plasma generators, the series comprising a first non-thermal plasma generator and at least a second non-thermal plasma generator, to give rise to a plasma-treated gaseous medium,
wherein the plasma-treated gaseous medium of at least one of the non-thermal plasma generator is discharged to the entrance of at least one other non-thermal plasma generator; and
discharging the plasma-treated gaseous medium from the last in the series of the fluidically-coupled non-thermal plasma generators.

22. A method for inactivating biologic agents in a gaseous medium, comprising:
directing a gaseous medium comprising biological agents through an entrance port of a dielectric barrier discharge device;
contacting the gaseous medium with a non-thermal plasma generated by said dielectric barrier discharge device to give rise to a plasma-treated gaseous medium;
directing the plasma-treated gaseous medium through an exit port of the dielectric barrier discharge device; and
further directing the plasma-treated gaseous medium through an entrance port of at least one other dielectric barrier discharge device.

23. The method of claim 22, wherein said non-thermal dielectric barrier discharge is generated by an oscillating electrical pulse or continuous wave of about 1 kHz to about 20,000 kHz.

24. The method of claim 22, wherein said non-thermal dielectric barrier discharge is generated by a high frequency electrical oscillation of about 5 kHz to about 30 kHz.

25. The method of claim 23, wherein said high frequency oscillation is generated by applying a voltage of about 1 kV to about 50 kV to the gaseous medium.

26. The method of claim 23, wherein said high frequency oscillation is generated by applying a voltage of about 5 kV to about 30 kV to the gaseous medium.
